(19) United States
(12) Patent Application Publication
(10) Pub. No.: US 2025/0098777 A1
(43) Pub. Date: Mar. 27, 2025

(54) ULTRASONIC VIBRATION-BASED AEROSOL GENERATING DEVICE AND CONTROL METHOD THEREOF

(71) Applicant: KT&G CORPORATION, Daejeon (KR)

(72) Inventors: Jang Won SEO, Daejeon (KR); Gyung Min GO, Daejeon (KR); Chul Ho JANG, Bucheon-si (KR); Jin Chul JUNG, Daejeon (KR)

(73) Assignee: KT&G CORPORATION, Daejeon (KR)

(21) Appl. No.: 18/569,071

(22) PCT Filed: Oct. 5, 2023
(86) PCT No.: PCT/KR2023/015285

§ 371 Date: Dec. 11, 2023

(30) Foreign Application Priority Data
Oct. 7, 2022 (KR) 10-2022-0128906

(51) Int. Cl. A24F 40/50; A24F 40/05; A24F 40/10; A24F 40/65; G06F 21/31

(52) U.S. Cl. CPC A24F 40/50; A24F 40/05; A24F 40/10; A24F 40/65; G06F 21/31

(57) ABSTRACT

Provided is an aerosol generating device which determines a type of a liquid included in a cartridge, transmits a signal identifying the type of the liquid to a terminal, receives a control signal including information about the liquid from the terminal, controls an operation of the electronic device based on the control signal, and transmits an inhalation completion notification to the terminal.

### TECHNICAL FIELD

The following embodiments relate to a device for generating an aerosol, and more particularly, to a method of controlling inhalation of an aerosol generating device.

### BACKGROUND ART

The demand for electronic cigarettes, or e-cigarettes, has recently been on the rise. The rising demand for e-cigarettes has accelerated the continued development of e-cigarette-related functions, such as functions according to the types and characteristics of e-cigarettes.

### DISCLOSURE OF THE INVENTION

### Technical Goals

An embodiment may provide an aerosol generating device for generating an aerosol.

Another embodiment may provide a method of controlling inhalation of an aerosol generating device.

### Technical Solutions

A method of controlling a terminal according to an embodiment includes receiving, from an electronic device, information about a cartridge liquid of the electronic device, transmitting a request for information about the cartridge liquid to a server, receiving the information about the cartridge liquid from the server, transmitting a control signal comprising the information about the cartridge liquid to the electronic device, receiving an inhalation completion notification from the electronic device, and updating operating condition information of the electronic device in response to the inhalation completion notification.

The method may further include, displaying the information about the cartridge liquid on the terminal.

The updating of the operating condition information may include determining a next inhalation start time of the cartridge liquid, and restricting an operation of the electronic device until the determined next time point.

The method may further include performing user authentication when the information about the cartridge liquid comprises information about inhalation restriction.

The performing of the user authentication may include controlling the electronic device to cease the operation until the user authentication is completed.

The information about the cartridge liquid may indicate at least one of a puffing time, a puffing cycle, and a number of puffs, which are required for one dose of the cartridge liquid.

A method of controlling an electronic device according to an embodiment includes determining a type of a liquid included in a cartridge, transmitting a signal identifying the type of the liquid to a terminal, in response to the signal, receiving a control signal including information about the liquid from the terminal, controlling an operation of the electronic device based on the control signal, and transmitting an inhalation completion notification to the terminal when inhalation of one dose of the liquid is completed.

The controlling of the operation of the electronic device may include providing a puff-inducing interface based on the control signal.

### Effects

A method of controlling inhalation of an aerosol generating device according to embodiments may be provided.

An aerosol generating device for generating an aerosol according to embodiments may be provided.

### BRIEF DESCRIPTION OF DRAWINGS

FIG. 1 is a block diagram of an aerosol generating device according to an example.

FIG. 2 is a schematic view of an aerosol generating device according to an example.

FIG. 3 is a perspective view illustrating that a cartridge and a body of an aerosol generating device are separated according to an example.

FIG. 4 is a perspective view illustrating that a cartridge and a body of an aerosol generating device are coupled according to an example.

FIG. 5 is a flowchart illustrating a method of controlling a terminal according to an embodiment.

FIG. 6 is a flowchart illustrating a method of controlling an aerosol generating device according to an embodiment.

### BEST MODE FOR CARRYING OUT THE INVENTION

The following detailed structural or functional description is provided as an example only and various alterations and modifications may be made to the embodiments. Here, the embodiments are not construed as limited to the disclosure and should be understood to include all changes, equivalents, and replacements within the idea and the technical scope of the disclosure.

Although terms of “first,” “second,” and the like are used to explain various components, the components are not limited to such terms. These terms are used only to distinguish one component from another component. For example, a first component may be referred to as a second component, or similarly, the second component may be referred to as the first component within the scope of the present disclosure.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

As used herein, “A or B”, “at least one of A and B”, “at least one of A or B”, “A, B or C”, “at least one of A, B and C”, and “at least one of A, B, or C” may include any one of the items listed together in the corresponding one of the phrases, or all possible combinations thereof.

Unless otherwise defined, all terms, including technical and scientific terms, used herein have the same meaning as commonly understood by one of ordinary skill in the art to which the present disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, the embodiments will be described in detail with reference to the accompanying drawings. When describing the embodiments with reference to the accompanying drawings, like reference numerals refer to like elements and a repeated description related thereto will be omitted.

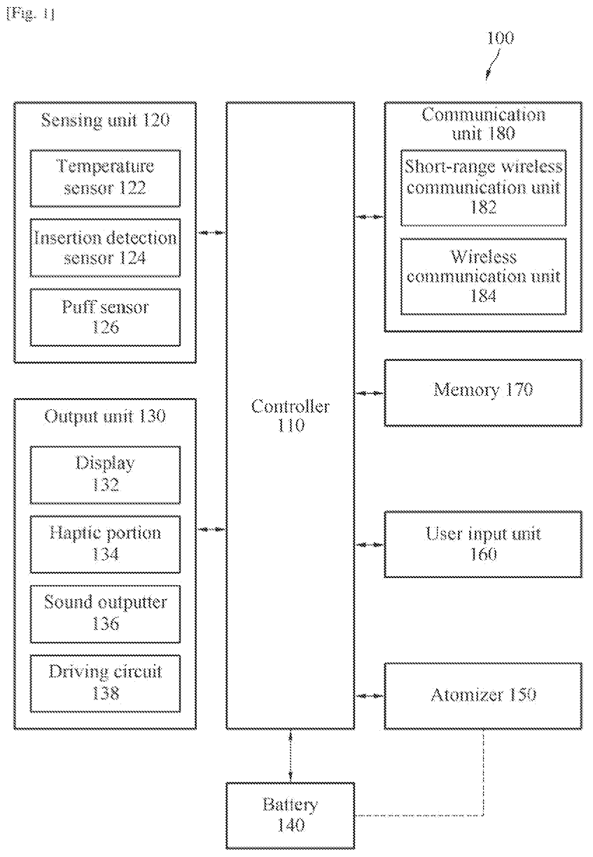

FIG. 1 is a block diagram of an aerosol generating device according to an embodiment.

According to an embodiment, an aerosol generating device 100 of FIG. 1 may include a controller 110, a sensing unit 120, an output unit 130, a battery 140, an atomizer 150, a user input unit 160, a memory 170, and a communication unit 180. However, an internal structure of the aerosol generating device 100 is not limited to what is shown in FIG. 1. It is to be understood by one of ordinary skill in the art to which the disclosure pertains that some of the components shown in FIG. 1 may be omitted or new components may be added according to the design of the aerosol generating device 100.

The sensing unit 120 may sense a state of the aerosol generating device 100 or a state of an environment around the aerosol generating device 100, and transmit sensing information obtained through the sensing to the controller 110. Based on the sensing information, the controller 110 may control the aerosol generating device 100 to control operations of the atomizer 150, restrict smoking, determine whether an aerosol generating article (e.g., an aerosol generating article, a cartridge, etc.) is inserted, display a notification, and perform other functions.

The sensing unit 120 may include at least one of a temperature sensor 122, an insertion detection sensor 124, or a puff sensor 126. However, embodiments are not limited thereto.

The temperature sensor 122 may sense a temperature of the atomizer 150 (or an aerosol generating material). The aerosol generating device 100 may include a separate temperature sensor for sensing a temperature of the atomizer 150, or the atomizer 150 itself may perform a function as a temperature sensor. Alternatively, the temperature sensor 122 may be arranged around the battery 140 to monitor a temperature of the battery 140.

The insertion detection sensor 124 may sense whether the aerosol generating article is inserted and/or removed. The insertion detection sensor 124 may include, for example, at least one of a film sensor, a pressure sensor, a light sensor, a resistive sensor, a capacitive sensor, an inductive sensor, or an infrared sensor, which may sense a signal change by the insertion and/or removal of the aerosol generating article.

The puff sensor 126 may sense a puff from a user based on various physical changes in an airflow path or airflow channel. For example, the puff sensor 126 may sense the puff from the user based on one of a temperature change, a flow change, a voltage change, and a pressure change.

The sensing unit 120 may further include at least one of a temperature/humidity sensor, an atmospheric pressure sensor, a magnetic sensor, an acceleration sensor, a gyroscope sensor, a position sensor (e.g., a global positioning system (GPS)), a proximity sensor, or a red, green, blue (RGB) sensor (e.g., an illuminance sensor), in addition to the sensors 122 to 126 described above. A function of each sensor may be intuitively inferable from its name by one of ordinary skill in the art, and thus, a more detailed description thereof will be omitted here.

The output unit 130 may output information about the state of the aerosol generating device 100 and provide the information to the user. The output unit 130 may include at least one of a display 132, a haptic portion 134, or a sound outputter 136. However, embodiments are not limited thereto. When the display 132 and a touchpad are provided in a layered structure to form a touchscreen, the display 132 may be used as an input device in addition to an output device.

The display 132 may visually provide the information about the aerosol generating device 100 to the user. The information about the aerosol generating device 100 may include, for example, a charging/discharging state of the battery 140 of the aerosol generating device 100, a state of the atomizer 150, an insertion/removal state of the aerosol generating article, a limited usage state (e.g., an abnormal article detected) of the aerosol generating device 100, or the like, and the display 132 may externally output the information. The display 132 may be, for example, a liquid-crystal display panel (LCD), an organic light-emitting display panel (OLED), or the like. The display 132 may also be in the form of a light-emitting diode (LED) device.

The haptic portion 134 may provide the information about the aerosol generating device 100 to the user in a haptic way by converting an electrical signal into a mechanical stimulus or an electrical stimulus. The haptic portion 134 may include, for example, a motor, a piezoelectric element, or an electrical stimulation device.

The sound outputter 136 may provide the information about the aerosol generating device 100 to the user in an auditory way. For example, the sound outputter 136 may convert an electrical signal into a sound signal and externally output the sound signal.

The battery 140 may supply power to be used to operate the aerosol generating device 100. The battery 140 may supply power to operate the atomizer 150. In addition, the battery 140 may supply power required for operations of the other components (e.g., the sensing unit 120, the output unit 130, the user input unit 160, the memory 170, and the communication unit 180) included in the aerosol generating device 100. The battery 140 may be a rechargeable battery or a disposable battery. The battery 140 may be, for example, a lithium polymer (LiPoly) battery. However, embodiments are not limited thereto.

The atomizer 150 may receive power from the battery 140 to atomize the aerosol generating material. Although not shown in FIG. 1, the aerosol generating device 100 may further include a power conversion circuit (e.g., a direct current (DC)-to-DC (DC/DC) converter) that converts power of the battery 140 and supplies the power to the atomizer 150. In addition, when the aerosol generating device 100 generates an aerosol by an ultrasonic vibrating method, the aerosol generating device 100 may further include a DC-to-alternating current (AC) (DC/AC) converter that converts DC power of the battery 140 into AC power.

The controller 110, the sensing unit 120, the output unit 130, the user input unit 160, the memory 170, and the communication unit 180 may receive power from the battery 140 to perform functions. Although not shown in FIG. 1, the aerosol generating device 100 may further include a power conversion circuit, for example, a low dropout (LDO) circuit or a voltage regulator circuit, which converts power of the battery 140 and supplies the power to respective components.

In an embodiment, the atomizer 150 may include a vibrator that generates ultrasonic vibrations by an applied signal (e.g., power). For example, a material of the vibrator may include a piezoelectric ceramic. However, embodiments are not limited thereto. The vibrator may include a piezoelectric body. The piezoelectric body according to an embodiment may be a conversion element that may convert electrical energy into mechanical energy and may generate an ultrasonic vibration under the control of the controller 110. In an embodiment, when AC power is applied to a piezoelectric body that is subjected to polarization processing, the piezoelectric body may repeatedly expand and contract. As the piezoelectric body repeatedly expands and contracts, the vibrator may vibrate at a characteristic frequency. As a signal is applied to the vibrator, a short high-frequency vibration may be generated, and the generated vibration may break the aerosol generating material into small particles and atomize the aerosol generating material into an aerosol.

The user input unit 160 may receive information input from the user or may output information to the user. For example, the user input unit 160 may include a keypad, a dome switch, a touchpad (e.g., a contact capacitive type, a pressure resistive film type, an infrared sensing type, a surface ultrasonic conduction type, an integral tension measurement type, a piezo effect method, etc.), a jog wheel, a jog switch, or the like. However, embodiments are not limited thereto. In addition, although not shown in FIG. 1, the aerosol generating device 100 may further include a connection interface such as a universal serial bus (USB) interface, and may be connected to another external device through the connection interface such as a USB interface to transmit and receive information or to charge the battery 140.

The memory 170, which is hardware for storing various pieces of data processed in the aerosol generating device 100, may store data processed by the controller 110 and data to be processed by the controller 110. The memory 170 may include at least one type of storage medium of a flash memory type memory, a hard disk type memory, a multimedia card micro type memory, a card type memory (e.g., an SD or XD memory), a random access memory (RAM), a static random access memory (SRAM), a read-only memory (ROM), an electrically erasable programmable read-only memory (EEPROM), a programmable read-only memory (PROM), a magnetic memory, a magnetic disk, or an optical disk. The memory 170 may store an operating time of the aerosol generating device 100, a maximum number of puffs, a current number of puffs, at least one temperature profile, data associated with a smoking pattern of the user, or the like.

The communication unit 180 may include at least one component for communicating with another electronic device. For example, the communication unit 180 may include a short-range wireless communication unit 182 and a wireless communication unit 184.

The short-range wireless communication unit 182 may include a Bluetooth communication unit, a Bluetooth low energy (BLE) communication unit, a near field communication unit, a wireless area network (WLAN) (wireless fidelity (Wi-Fi)) communication unit, a ZigBee communication unit, an infrared data association (IrDA) communication unit, a Wi-Fi direct (WFD) communication unit, an ultra-wideband (UWB) communication unit, and an Ant+ communication unit. However, embodiments are not limited thereto.

The wireless communication unit 184 may include, for example, a cellular network communication unit, an Internet communication unit, a computer network (e.g., a local area network (LAN) or a wide-area network (WAN)) communication unit, or the like. However, embodiments are not limited thereto. The wireless communication unit 184 may use subscriber information (e.g., international mobile subscriber identity (IMSI)) to identify and authenticate the aerosol generating device 100 in a communication network.

The controller 110 may control the overall operation of the aerosol generating device 100. In an embodiment, the controller 110 may include at least one processor. The processor may be implemented as an array of a plurality of logic gates, or may be implemented as a combination of a general-purpose microprocessor and a memory in which a program executable by the microprocessor is stored. In addition, it is to be understood by one of ordinary skill in the art to which the present disclosure pertains that it may be implemented in other types of hardware.

The controller 110 may control an operation of the atomizer 150 by controlling the supply of power from the battery 140 to the atomizer 150. For example, the controller 110 may control the supply of power by controlling switching of a switching element of a driving circuit 138 positioned between the battery 140 and the atomizer 150.

The controller 110 may analyze a sensing result obtained by the sensing of the sensing unit 120 and control processes to be performed thereafter. For example, the controller 110 may control power to be supplied to the atomizer 150 to start or end an operation of the atomizer 150 based on the sensing result obtained by the sensing unit 120. In another example, the controller 110 may control an amount of power to be supplied to the atomizer 150 and a time for which the power is to be supplied, such that the atomizer 150 may vibrate at a predetermined frequency or maintain a desired vibration frequency based on the sensing result obtained by the sensing unit 120.

The controller 110 may control the output unit 130 based on the sensing result obtained by the sensing unit 120. For example, when a number of puffs counted through the puff sensor 126 reaches a preset number, the controller 110 may inform the user that the aerosol generating device 100 is to be ended soon, through at least one of the display 132, the haptic portion 134, or the sound outputter 136.

In an embodiment, the controller 110 may control a power supply time and/or a power supply amount for the atomizer 150 by controlling the driving circuit 138 according to a state of the aerosol generating article sensed by the sensing unit 120. For example, the controller 110 may control a vibration frequency of the vibrator of the atomizer 150 according to the type or a remaining amount of the aerosol generating article.

An embodiment may be implemented in the form of a recording medium including instructions executable by a computer, such as a program module executable by the computer. A computer-readable medium may be any available medium that may be accessed by a computer and includes all of a volatile medium, a non-volatile medium, a removable medium, and a non-removable medium. In addition, the computer-readable medium may include both a computer storage medium and a communication medium. The computer storage medium includes all of a volatile medium, a non-volatile medium, a removable medium, and a non-removable medium implemented by any method or technology for storage of information such as computer-readable instructions, data structures, program modules or other data. The communication medium typically includes a computer-readable command, a data structure, or other data regarding a modulated data signal such as a program module, or other transmission mechanisms, and includes an arbitrary information transfer medium.

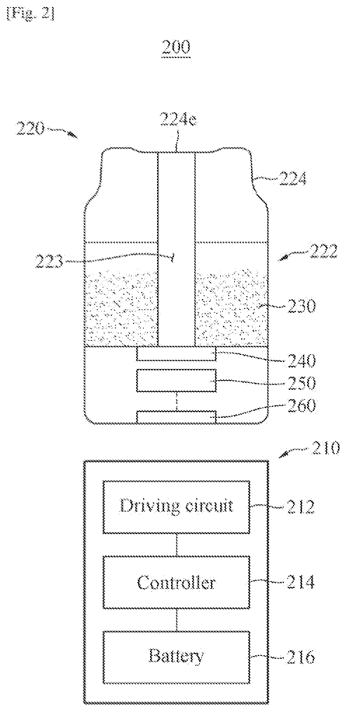

FIG. 2 is a schematic diagram of an aerosol generating device according to an embodiment.

Referring to FIG. 2, an aerosol generating device 200 (e.g., the aerosol generating device 100 of FIG. 1) may include a cartridge 220 containing an aerosol generating material and a body 210 connected to the cartridge 220.

The cartridge 220 of the aerosol generating device 200 may be coupled to the body 210 while accommodating the aerosol generating material therein. For example, as at least a portion of the cartridge 220 is inserted into the body 210, the cartridge 220 and the body 210 may be coupled. In another example, as at least a portion of the body 210 is inserted into the cartridge 220, the cartridge 220 and the body 210 may be coupled.

The cartridge 220 and the body 210 may be coupled by at least one of a snap-fit method, a screw coupling method, a magnetic coupling method, or an interference fit method, but the coupling method of the cartridge 220 and the body 210 is not limited to the above examples.

According to an embodiment, the cartridge 220 may include a housing 222, a mouthpiece 224, a storage portion 230, a transfer portion 240, a vibrator 250, and an electrical terminal 260.

The housing 222 of the aerosol generating device 200 may form the overall appearance of the cartridge 220 together with the mouthpiece 224, and components for an operation of the cartridge 220 may be disposed inside the housing 222. For example, the housing 222 may be formed in a rectangular parallelepiped shape, but the shape of the housing 222 is not limited to the embodiment described above. According to an embodiment, the housing 222 may be formed in the shape of a polygonal column (e.g., a triangular column or a pentagonal column) or a cylindrical column.

The mouthpiece 224 of the aerosol generating device 200 may be disposed in one area of the housing 222 and may include an outlet 224e for discharging an aerosol generated from an aerosol generating material to the outside. For example, the mouthpiece 224 may be disposed in an area of the cartridge 220 opposite to another area coupled to the body 210, and the user may receive an aerosol from the cartridge 220 as the user brings the mouth into contact with the mouthpiece 224 and inhales the aerosol.

A pressure difference may occur between the outside of the cartridge 220 and the inside of the cartridge 220 due to a user's inhalation or puff operation, and an aerosol generated in the cartridge 220 may be discharged to the outside of the cartridge 220 through the outlet 224e due to the pressure difference between the inside and the outside of the cartridge 220. That is, the user may receive the aerosol discharged to the outside of the cartridge 220 through the outlet 224e as the user brings the mouth into contact with the mouthpiece 224 and inhales the aerosol.

The storage portion 230 of the aerosol generating device 200 may be positioned in an inner space of the housing 222 and may contain an aerosol generating material. In the present disclosure, the expression “the storage portion contains the aerosol generating material” means that the storage portion 230 performs a function of simply containing an aerosol generating material, such as the use of a container, and the storage portion 230 includes an element that impregnates (contains) an aerosol generating material, such as a sponge, cotton, cloth, or porous ceramic structure therein. In addition, the above expression may be used as the same meaning below.

The storage portion 230 may contain an aerosol generating material in one of a liquid state, a solid state, a gaseous state, and a gel state.

In an embodiment, the aerosol generating material may include a liquid composition. The liquid composition may be, for example, a liquid including a tobacco-containing material that includes a volatile tobacco flavor component, or may be a liquid including a non-tobacco material.

The liquid composition may include, for example, one of water, a solvent, ethanol, a plant extract, a fragrance, a flavoring agent, or a vitamin mixture, or a mixture these ingredients. The fragrance may include, for example, menthol, peppermint, spearmint oil, various fruit-flavored ingredients, and the like. However, embodiments are not limited thereto.

The flavoring agent may include ingredients that provide the user with a variety of flavors or scents. The vitamin mixture may be a mixture of at least one of vitamin A, vitamin B, vitamin C, or vitamin E. However, embodiments are not limited thereto. The liquid composition may also include an aerosol former such as glycerin and propylene glycol.

The liquid composition may include, for example, glycerin and propylene glycol in any weight ratio, to which a nicotine salt is added. The liquid composition may also include two or more types of nicotine salt. A nicotine salt may be formed by adding a suitable acid including an organic acid or an inorganic acid to nicotine. The nicotine may be either naturally generated nicotine or synthetic nicotine and may have a concentration of any appropriate weight relative to a total solution weight of the liquid composition.

The acid for forming the nicotine salt may be appropriately selected in consideration of an absorption rate of nicotine in the blood, an operating temperature of the aerosol generating device 200, a flavor or taste, solubility, and the like. For example, the acid for forming the nicotine salt may include a single acid selected from the group consisting of a benzoic acid, lactic acid, salicylic acid, lauric acid, sorbic acid, levulinic acid, pyruvic acid, formic acid, acetic acid, propionic acid, butyric acid, valeric acid, caproic acid, caprylic acid, capric acid, citric acid, myristic acid, palmitic acid, stearic acid, oleic acid, linoleic acid, linolenic acid, phenylacetic acid, tartaric acid, succinic acid, fumaric acid, gluconic acid, saccharic acid, malonic acid, or malic acid, or a mixture of two or more acids selected from the above group. However, embodiments are not limited thereto.

The transfer portion 240 of the aerosol generating device 200 may absorb an aerosol generating material. For example, the aerosol generating material stored or contained in the storage portion 230 may be transferred from the storage portion 230 to the vibrator 250 through the transfer portion 240, and the vibrator 250 may generate an aerosol by atomizing the aerosol generating material of the transfer portion 240 or the aerosol generating material received from the transfer portion 240. In this case, the transfer portion 240 may include at least one of cotton fibers, ceramic fibers, glass fibers, or porous ceramics, but the transfer portion 240 is not limited to the embodiment described above.

According to an embodiment, the transfer portion 240 may be disposed adjacent to the storage portion 230 to receive a liquid aerosol generating material from the storage portion 230. For example, the aerosol-generating material stored in the storage portion 230 may be discharged to the outside of the storage portion 230 through a liquid supply port formed in one area of the storage portion 230 facing toward the transfer portion 240, and the transfer portion 240 may absorb the aerosol-generating material discharged from the storage portion 230.

According to an embodiment, the cartridge 220 may further include an absorber that is disposed to cover at least a portion of the vibrator 250 where an aerosol is generated, and transfers the aerosol generating material absorbed by the transfer portion 240 to the vibrator 250. The absorber may be made of a material capable of absorbing an aerosol generating material. For example, the absorber may include at least one material of SPL 30(H), SPL 50(H) V, NP 100(V8), SPL 60 (FC), and melamine. As the cartridge 220 further includes the absorber, the aerosol generating material may be absorbed not only in the transfer portion 240 but also in the absorber, so that the amount of aerosol generating material being absorbed may improve.

The vibrator 250 of the aerosol generating device 200 may be positioned inside the housing 222 and may generate an aerosol by converting a phase of the aerosol generating material stored in the cartridge 220. For example, the vibrator 250 may generate an aerosol by heating or vibrating an aerosol generating material.

In addition, as the absorber is disposed to cover at least a portion of the vibrator 250, the absorber may function as a physical barrier to prevent “spitting” such that particles that are not sufficiently atomized during the aerosol generating process are not discharged directly to the outside of the aerosol generating device 200. Here, “spitting” may indicate that particles of an aerosol generating material having relatively large sizes as not sufficiently atomized are discharged to the outside of the cartridge 220. As the cartridge 220 further includes the absorber, the possibility of spitting may be reduced, and the smoking satisfaction of the user may improve.

In an embodiment, the absorber may be positioned between one surface of the vibrator 250 where an aerosol is generated and the transfer portion 240, and transfer the aerosol supplied to the transfer portion 240 to the vibrator 250. For example, one area of the absorber may contact the bottom surface of the transfer portion 240, and another area of the absorber may contact the top surface of the vibrator 250. That is, the absorber may be positioned on a top surface of the vibrator 250, and supply the aerosol generating material absorbed by the transfer portion 240 to the vibrator 250.

According to an embodiment, the vibrator 250 of the aerosol generating device 200 may change a phase of the aerosol generating material by using an ultrasonic vibrating method that atomizes the aerosol generating material with ultrasonic vibration. For example, the vibrator 250 may generate vibration of a short period, and the vibration generated from the vibrator 250 may be ultrasonic vibration. A frequency of the ultrasonic vibration may be in a range of about 100 kilohertz (kHz) to about 10 megahertz (MHz) (preferably, a range of about 100 kHz to 3.5 MHz). However, embodiments are not limited thereto. As the vibrator generates ultrasonic vibration of the frequency band described above, the vibrator may vibrate in a longitudinal direction (e.g., a z-axis direction) of the cartridge 220 or the housing 222. However, embodiments are not limited to the direction in which the vibrator vibrates, and the direction in which the vibrator vibrates may be changed to various directions (e.g., one of an x-axis direction, a y-axis direction, and the z-axis direction or a combination thereof). The aerosol generating material supplied from the storage portion 230 to the vibrator 250 by the vibration of the short period generated from the vibrator 250 may be vaporized and/or change into particles to be atomized into an aerosol.

For example, the vibrator 250 may include a piezoelectric ceramic, and the piezoelectric ceramic may be a functional material capable of converting power and a mechanical force into each other by generating power (a voltage) by a physical force (a pressure) and generating vibration (a mechanical force) when the power is applied thereto. That is, as power is applied to the vibrator 250, the vibration of the short period (the physical force) may be generated, and the generated vibration may break the aerosol generating material into small particles and atomize the aerosol generating material into an aerosol.

The vibrator 250 may be electrically connected to other components of the aerosol generating device 200 through the electrical terminal 260. The electrical terminal 260 may be positioned on one surface of the cartridge 220. For example, the electrical terminal 260 may be positioned on a coupling surface of the cartridge 220 where the cartridge 220 is coupled to the body 210 of the aerosol generating device 20. The electrical terminal 260 may be positioned on one surface of the housing 222 opposite the mouthpiece 224.

According to an embodiment, the vibrator 250 may be electrically connected to at least one of a driving circuit 212, a controller 214, or a battery 216 of the body 210 through the electrical terminal 260 positioned inside the housing 222 of the cartridge 220.

For example, the vibrator 250 may be electrically connected to the electrical terminal 260 positioned inside the cartridge 220 through a first conductor, and the electrical terminal 260 may be electrically connected to the driving circuit 212 of the body 210 through a second conductor. That is, the vibrator 250 may be electrically connected to components of the body 210 through the electrical terminal 260.

The vibrator 250 may generate ultrasonic vibration by receiving power from the battery 216 of the body 210 through the electrical terminal 260. In addition, the vibrator 250 may be electrically connected to the controller 214 of the body 210 through the electrical terminal 260, and the controller 214 may control the operation of the vibrator 250 through the driving circuit 212.

For example, the electrical terminal 260 may include at least one of a pogo pin, a wire, a cable, a printed circuit board (PCB), a flexible printed circuit board (FPCB), or a C-clip. However, the electrical terminal 260 is not limited to the above examples.

In an embodiment, the vibrator 250 may be implemented as a mesh-shaped or plate-shaped vibration accommodation portion that performs both a function of absorbing an aerosol generating material and maintaining the aerosol generating material in an optimal state to be converted into an aerosol and a function of transferring vibration to the aerosol generating material to generate an aerosol, without using the separate transfer portion 240.

The aerosol generated by the vibrator 250 may be discharged to the outside of the cartridge 220 through an airflow path 223 and supplied to the user.

According to an embodiment, the airflow path 223 may be positioned inside the cartridge 220 and may be connected to the vibrator 250 and the outlet 224e of the mouthpiece 224. Accordingly, the aerosol generated by the vibrator 250 may flow along the airflow path 223 and may be discharged to the outside of the cartridge 220 or the aerosol generating device 200 through the outlet 224e. The user may receive the aerosol as the user brings the mouth into contact with the mouthpiece 224 and inhales the aerosol discharged from the outlet 224e.

Although not shown in the drawings, the airflow path 223 may include at least one inlet through which air outside the cartridge 220 is introduced into the cartridge 220. The inlet may be positioned on at least a portion of the housing 222 of the cartridge 220. For example, the inlet may be positioned on the coupling surface (e.g., a bottom surface) of the cartridge 220 where the cartridge 220 and the body 210 are coupled.

Since at least one gap may be formed in a portion where the cartridge 220 and the body 210 are coupled, external air may be introduced through the gap between the cartridge 220 and the body 210 and move into the cartridge 220 through the inlet.

The airflow path 223 may be connected from the inlet to a space where an aerosol is generated by the vibrator 250, and may be connected from the corresponding space to the outlet 224e.

Accordingly, the air introduced through the inlet may be transferred to the vibrator 250, and the transferred air may move to the outlet 224e together with the aerosol generated by the vibrator 250, thereby circulating the air inside the cartridge 220.

According to an embodiment, at least a portion of the airflow path 223 may be disposed such that an outer circumferential surface is surrounded by the storage portion 230 in the housing 222. In another example, at least a portion of the airflow path 223 may be disposed between an inner wall of the housing 222 and an outer wall of the storage portion 230. The arrangement structure of the airflow path 223 is not limited to the above examples, and the airflow path 223 may be arranged in various structures to circulate the airflow between the inlet, the vibrator 250, and the outlet 224e.

According to an embodiment, the body 210 may include the driving circuit 212, the controller 214, and the battery 216 therein, and one end portion of the body 210 may be connected to one end portion of the cartridge 220. For example, the body 210 may be coupled to the bottom surface or the coupling surface of the cartridge 220.

When the vibrator 250 of the cartridge 220 is electrically connected to the driving circuit 212 through the electrical terminal 260, the driving circuit 212 may supply power to the vibrator 250. For example, a magnitude of power supplied to the vibrator 250 may be determined by the controller 214. A vibration frequency of the vibrator 250 or the like may be controlled by the magnitude of the power. The driving circuit 212 according to an embodiment may be in the form of a Class-E power amplifier circuit, a half bridge circuit, or a full bridge circuit. However, embodiments are not limited to the described embodiment.

The controller 214 may control the overall operation of the aerosol generating device 200. For example, the controller 214 may control the amount of aerosol generated by the vibrator 250 by controlling power supplied from the battery 216 to the vibrator 250. For example, the controller 214 may control power supplied to the vibrator 250 so that the vibrator 250 may vibrate at a predetermined frequency.

The controller 214 may be implemented as an array of a plurality of logic gates, or may be implemented as a combination of a general-purpose microprocessor and a memory in which a program executable by the microprocessor is stored. In addition, it is to be understood by one of ordinary skill in the art to which the disclosure pertains that the controller 214 may be implemented in other types of hardware.

The controller 214 analyzes a sensing result obtained by at least one sensor included in the aerosol generating device 200 and controls subsequent processes to be performed. For example, the controller 214 may control power to be supplied to the vibrator 250 to start or end an operation of the vibrator 250 based on the sensing result obtained by the at least one sensor. In addition, the controller 214 may control an amount of power to be supplied to the vibrator 250 and a time for which the power is to be supplied, such that the vibrator 250 may generate an appropriate amount of aerosol based on the sensing result obtained by the at least one sensor.

The battery 216 may supply power to be used to operate the aerosol generating device 200. For example, when the body 210 is electrically coupled to the cartridge 220, the battery 216 may supply power to the vibrator 250.

The battery 216 may supply power required for operations of the other hardware components (e.g., a sensor, a user interface, a memory, and the controller 214) included in the aerosol generating device 200. The battery 216 may be a rechargeable battery or a disposable battery.

For example, the battery 216 may include a nickel-based battery (e.g., a nickel-metal hydride battery or a nickel-cadmium battery) or a lithium-based battery (e.g., a lithium-cobalt battery, a lithium-phosphate battery, a lithium-titanate battery, a lithium-ion battery, or a lithium-polymer battery).

In an embodiment, a cross-section of the aerosol generating device 200 taken in a direction transverse to the longitudinal direction of the cartridge 220 and/or the body 210 may be circular, elliptical, square, rectangular, or various polygonal shapes. However, the shape of the cross-section of the cartridge 220 and/or the body 210 is not limited to the above shapes. Also, the aerosol generating device 200 is not limited to a shape that linearly extends in the longitudinal direction.

In an embodiment, the aerosol generating device 200 may be curved or bent to make it easier for the user to hold by hand, and the shape of the cross-section of the aerosol generating device 200 may change along the longitudinal direction.

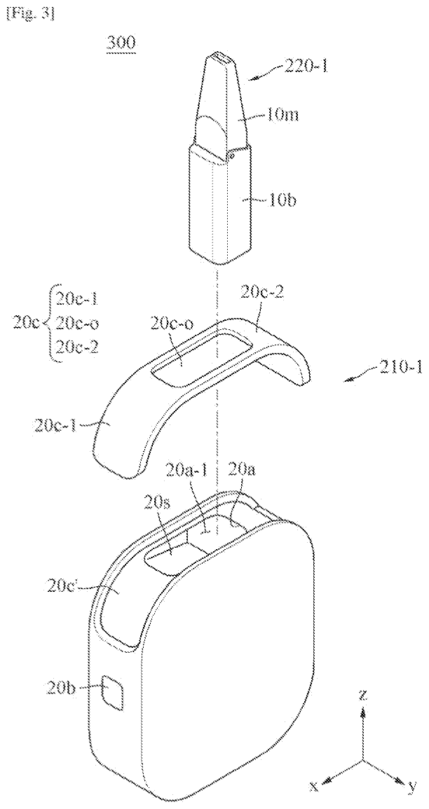

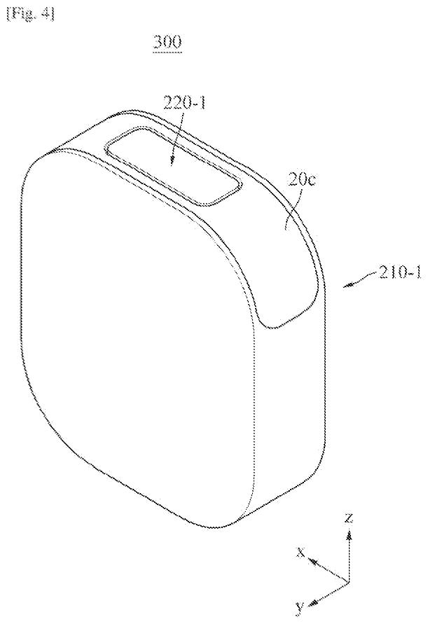

FIG. 3 is a perspective view illustrating that a cartridge and a body portion of an aerosol generating device are separated according to an embodiment, and FIG. 4 is a perspective view illustrating that a cartridge and a body portion of an aerosol generating device are coupled according to an embodiment.

An aerosol generating device 300 according to an embodiment shown in FIGS. 3 and 4 may be a modified example of the aerosol generating device 200 shown in FIG. 2 (or the aerosol generating device 100 of FIG. 1), and a cartridge 220-1 and a body 210-1 according to the embodiment shown in FIGS. 3 and 4 may be modified examples of the cartridge 220 and the body 210 shown in FIG. 2, respectively, and therefore, the repeated description will be omitted below.

Referring to FIGS. 3 and 4, the cartridge 220-1 may be detachably coupled to the body 210-1. For example, as at least a portion of the cartridge 220-1 is inserted into the body 210-1, the cartridge 220-1 may be coupled to the body 210-1.

The cartridge 220-1 may include a mouthpiece 10m that may move between an open position and a closed position. For example, the mouthpiece 10m may be opened and closed by rotating between the open position and the closed position.

A body portion 10b of the cartridge 220-1 may be coupled to the mouthpiece 10m through a rotation shaft. In an example, the mouthpiece 10m may be positioned at the open position. The open state of the mouthpiece 10m may refer to a state where the mouthpiece 10m is stretched in the longitudinal direction of the cartridge 220-1 to make it easier for the user to bring the mouth into contact with the mouthpiece 10m. Here, the longitudinal direction may refer to a direction in which the cartridge 220-1 extends the longest among several directions. In another example, the mouthpiece 10m may be positioned at the closed position. The closed state of the mouthpiece 10m may refer to a state where the mouthpiece 10m is folded in a direction transverse to the longitudinal direction of the cartridge 220-1 so that the mouthpiece 10m is accommodated in the body 210-1 of the aerosol generating device 300.

The cartridge 220-1 may include the body portion 10b including various components required to generate an aerosol and discharge the generated aerosol. For example, the body portion 10b may include at least a portion of each of a storage portion, a vibrator, and an airflow path.

The body 210-1 may include a coupling portion 20a to which the cartridge 220-1 is able to be coupled. For example, the body 210-1 may include an accommodation groove 20a-1 in which at least a portion of the cartridge 220-1 may be accommodated. The body portion 10b of the cartridge 220-1 may be inserted into the accommodation groove 20a-1. For example, the body portion 10b of the cartridge 220-1 may have a substantially rectangular column shape, and corners of the rectangular column may be chamfered or rounded. However, the shape of the body portion 10b of the cartridge 220-1 is not limited to the above examples and may be a cylindrical or polygonal column shape.

As described above with reference to FIG. 2, the cartridge 220-1 and the body 210-1 may be coupled by at least one of a snap-fit method, a screw coupling method, a magnetic coupling method, or an interference fit method. For example, the cartridge 220-1 may include a first magnetic body and the body 210-1 may include a second magnetic body so that the cartridge 220-1 and the body 210-1 may be coupled by a magnetic force. However, the intensity of the first magnetic material and the second magnetic material may be designed considering the ease of attachment and detachment of the cartridge 220-1 and the body 210-1 and/or operational stability of the aerosol generating device 300.

The body 210-1 may include a button 20b. The button 20b may be positioned on one surface of the body 210-1. For example, the button 20b may be positioned on one surface of the body 210-1 corresponding to one end 20c-1 of a cover 20c. The user may control the operation of the aerosol generating device 300 using the button 20b when using the aerosol generating device 300.

The body 210-1 may further include an accommodation portion 20s capable of accommodating the mouthpiece 10m of the cartridge 220-1 when the mouthpiece 10m moves to the closed position. The accommodation portion 20s may be positioned on one surface of the body 210-1 and may have a shape or size corresponding to that of the mouthpiece 10m.

As shown in FIG. 4, the mouthpiece 10m, which has moved to the closed position, may minimize a portion of the aerosol generating device 300 protruding outside, that is, a portion protruding outside from an outer surface of the body 210-1 at the closed position, thereby improving portability.

In an embodiment, the body 210-1 may further include the cover 20c coupled to a portion of the body 210-1. The cover 20c may be coupled to at least one surface of the body 210-1. For example, the cover 20c may be coupled to one side of the body 210-1 where the coupling portion 20a is positioned. Also, the cover 20c may be coupled to one side of the body 210-1 where the accommodation portion 20s is positioned.

The cover 20c may include an opening 20c-o. The cover 20c may include the opening 20c-o having a size corresponding to that of the mouthpiece 10m. For example, the opening 20c-o may have a predetermined length and width. Here, the width of the opening 20c-o may be smaller than or equal to that of a body of the cartridge 220-1 and may be larger than or equal to that of the mouthpiece 10m. A length of the opening 20c-o may be longer than or equal to that of the mouthpiece 10m.

The cover 20c may extend from one end 20c-1 to the other end 20c-2 to be disposed on a seating portion 20c′ of the body 210-1. For example, the seating portion 20c′ may have a size and shape corresponding to those of the cover 20c. The seating portion 20c′ may be a portion that extends in both directions from an inlet side of the coupling portion 20a and the accommodation portion 20s and is grooved to a predetermined depth so that the cover 20c is able to be coupled thereto.

When the cartridge 220-1 is coupled to the body 210-1, the cover 20c may be coupled to the body 210-1 after the cartridge 220-1 is coupled to the body 210-1. The cover 20c may be coupled to one side of the body 210-1 by at least one of a snap-fit method, an interference fit method, or a magnetic coupling method. However, embodiments are not limited thereto.

Since the cover 20c includes the opening 20c-o through which the mouthpiece 10m may pass, it is possible to protect the cartridge 220-1 without interfering the opening and closing motion of the mouthpiece 10m in a state where the cartridge 220-1 is coupled to the body 210-1, and maintain the coupling of the cartridge 220-1 and the body 210-1.

FIG. 4 shows the aerosol generating device 300 in which both the cartridge 220-1 and the cover 20c are coupled to the body 210-1 and the mouthpiece 10m is positioned at the closed position. As shown in the drawing, as the body 210-1 includes the accommodation portion 20s having a size and shape corresponding to those of the mouthpiece 10m, and the seating portion 20c′ having a size and shape corresponding to those of the cover 20c, and the cover 20c includes the opening 20c-o having a size and shape corresponding to those of the mouthpiece 10m, the overall finish of the aerosol generating device 300 is solid and smooth.

When the cartridge 220-1 is separated from the body 210-1, the cover 20c may be first separated from the body 210-1 and then the cartridge 220-1 may be separated from the body 210-1. As described above, the cover 20c and the cartridge 220-1 may be sequentially separated from the body 210-1 or sequentially coupled to the body 210-1.

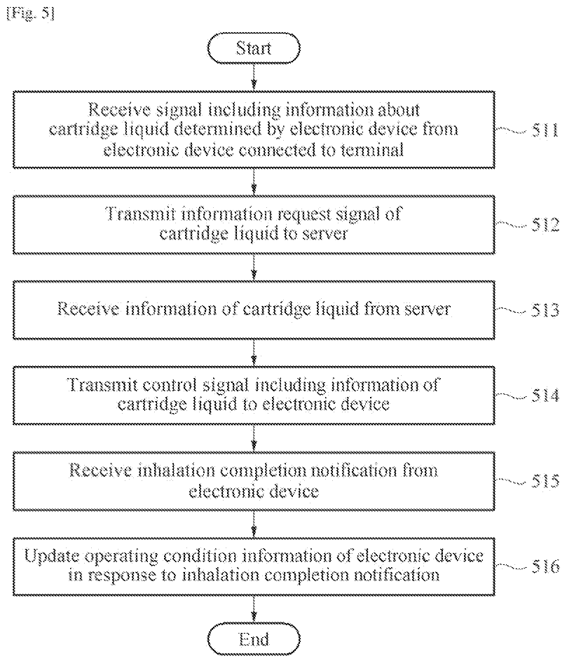

FIG. 5 is a flowchart illustrating a method of controlling a terminal according to an embodiment.

For ease of description, it will be described that operations 511 to 516 below are performed by a terminal that may communicate with the aerosol generating device 200 shown in FIG. 2. However, operations 511 to 516 may be performed by another suitable electronic device in a suitable system.

Furthermore, the operations of FIG. 5 may be performed in the shown order and manner. However, the order of some operations may be changed or omitted without departing from the spirit and scope of the shown example. The operations shown in FIG. 5 may be performed in parallel or simultaneously. Hereinafter, the aerosol generating device 200 according to an embodiment may be referred to as an electronic cigarette or an electronic device.

A terminal according to an embodiment is an electronic terminal capable of transmitting and receiving data with another device through wired or wireless communication, and may be portable. For example, the terminal may include a smartphone, a tablet, a personal digital assistant (PDA), and a portable multimedia player (PMP).

According to an embodiment, the aerosol generating device 200 may have functions of a terminal.

Those skilled in the art related to the present embodiment may understand that the terminal includes a memory, a user input unit, a display, a communication unit, and a controller and may understand functions of the components. The description below will be made assuming a smartphone as a terminal, but this is only an example and is not intended to limit the type of terminal to a smartphone.

The terminal may transmit and receive data with the aerosol generating device 200 through a communication link. The terminal may perform an operation associated with the aerosol generating device 200 or output information related to the aerosol generating device 200 by using data received from the aerosol generating device 200. Since a user generally carries the terminal, additional functions associated with the aerosol generating device 200 may be provided to the user without spatial restrictions by the terminal.

The aerosol generating device 200 according to an embodiment may receive an aerosol generating material through an aerosol generating article (e.g., the cartridge 220). Hereinafter, for ease of description, the aerosol generating article will be described based on a cartridge, but is not necessarily limited thereto.

The aerosol generating material according to an embodiment may include components for mind and body stability or various pharmacological ingredients for treatment, as well as nicotine. However, unlike other aerosol generating materials, the components for mind and body stability and/or the pharmacological ingredients may have a specific puffing time, a puffing cycle, and a number of puffs that have been proved to maximize the effect. Accordingly, when an aerosol generating material including the components for mind and body stability and/or the pharmacological ingredients is provided to the user, the aerosol generating device 200 needs to cease operation for a certain period of time after one dose in order to prevent overdose, or notify the user of a start timing of a next inhalation cycle.

In operation 511, the terminal according to an embodiment may receive a signal including information about a cartridge liquid determined by the aerosol generating device 200 from the aerosol generating device 200 connected to the terminal. Hereinafter, the term “liquid” refers to a material in a liquid state.

As described above, the aerosol generating material may include a liquid composition and information about the liquid according to an embodiment may refer to information about the types and ratios of components constituting the liquid composition.

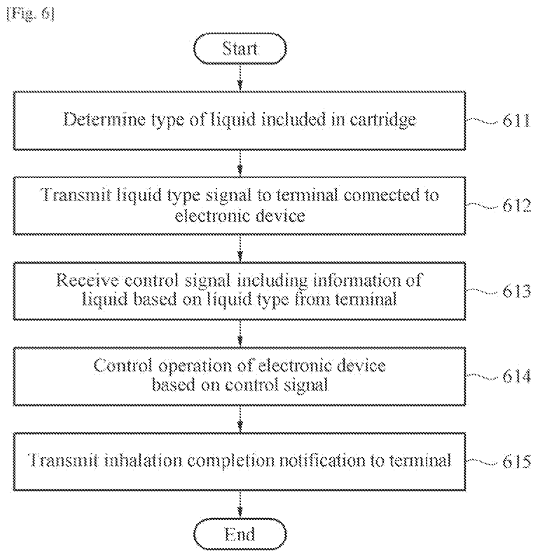

The aerosol generating device 200 according to an embodiment may determine liquid information of a cartridge inserted into the aerosol generating device 200. For example, when the aerosol generating device 200 determines that the inserted cartridge liquid is a first liquid (e.g., a nicotine-containing flavoring agent), the aerosol generating device 200 may transmit, to the connected terminal, information about the type of liquid indicating that the inserted cartridge liquid is the first liquid. A method of determining the liquid information of the cartridge by the aerosol generating device 200 according to an embodiment will be described in detail with reference to FIG. 6 below.

In operation 512, the terminal according to an embodiment may transmit an information request signal of the cartridge liquid to a server. For example, after the terminal receives the information about the type of the liquid (e.g., the first liquid) from the aerosol generating device 200, the terminal may request additional information about the first liquid from the server.

The information of the liquid of the cartridge requested to the server by the terminal may refer to information necessary for additional control related to the inhalation which may differ according to the liquid. For example, as described above, when the liquid includes components for mind and body stability and/or the pharmacological ingredients, whose use needs to be restricted, the aerosol generating device 200 may need additional control, and the terminal may request relevant information of the liquid from the server.

According to an embodiment, the information about the liquid of the cartridge may include at least one of a puffing time (i.e., a sum of durations of puffs), a puffing cycle (an interval between consecutive puffs), and a puffing number (i.e., the number of puffs), which are required for one dose of the liquid.

In operation 513, the terminal according to an embodiment may receive the information of the cartridge liquid from the server.

The terminal according to an embodiment may display the information of the cartridge liquid on the display of the terminal based on the received information of the cartridge liquid. For example, the type of the cartridge liquid, the puffing time required for one dose, the puffing cycle, and the puffing number, and the like may be displayed on the display of the terminal to derive the user to properly inhale.

The terminal is a device capable of installing and executing an application related to a server and the terminal may provide an interface to the user. The interface may be provided by the terminal itself. For example, the interface may be provided by an operating system (OS) of the terminal or may be provided by an application installed on the terminal. In addition, the interface may be provided by the server, and the terminal may simply receive and display the interface provided from the server.

According to an embodiment, when the terminal may obtain the information of the liquid without using the server (e.g., when the information of the liquid is stored in the memory of the terminal), operations 512 and 513 described above may not be performed.

The terminal may perform user authentication based on the received information of the cartridge liquid. When the information of the cartridge liquid includes information about inhalation restriction, the terminal may control the aerosol generating device 200 so that the cartridge liquid can be inhaled only after the user authentication is completed.

For example, the components for mind and body stability and/or the pharmacological ingredients may require a specific medication administration period and dosage tailored to the condition of the user. Therefore, individuals other than the designated user may need to be restricted from using the aerosol generating device 200. The terminal may perform the user authentication and allow the aerosol generating device 200 to operate only when the user is authenticated.

In operation 514, the terminal may transmit a control signal including the information of the cartridge liquid to the aerosol generating device 200. For example, the control signal may include signals related to a puffing time, a puffing cycle, and the number of puffs required for one dose. Also, the control signal may include a signal indicating that the user authentication is completed. In addition, the control signal may include a puff-inducing signal for smooth puffing of the user. The puff-inducing signal may be generated based on the signal related to the puffing time, the puffing cycle, and the puffing number required for one dose. The aerosol generating device 200 that has received the puff-inducing signal may provide various interfaces for inducing accurate puffs to the user.

In operation 515, the terminal may receive an inhalation completion notification from the aerosol generating device 200.

For example, when the user completes the inhalation of one dose of the cartridge liquid through the aerosol generating device 200, the aerosol generating device 200 may transmit a signal indicating that the operation for inhalation is completed to the terminal.

The inhalation completion notification may be determined based on the puffing time, the puffing cycle, and the puffing number required for one dose of the liquid. For example, when the puffing time required for one dose is reached, the aerosol generating device 200 may transmit a signal indicating that the operation for inhalation is completed to the terminal. Alternatively, when the puffing number required for one dose is reached, the aerosol generating device 200 may transmit a signal indicating that the operation for inhalation is completed to the terminal. However, the method of determining the inhalation completion notification is not limited to the above method. For example, when the user manually performs an operation of completing the inhalation (e.g., turning off the power of the aerosol generating device 200), the aerosol generating device 200 may transmit a signal indicating that the operation for inhalation is completed to the terminal at that time.

In operation 516, the terminal may update operating condition information of the aerosol generating device 200 in response to the inhalation completion notification. The operating condition information may include information for determining whether the aerosol generating device 200 should operate according to information about the cartridge liquid.

For example, when the cartridge liquid is a liquid including the mind and body stability and/or the pharmacological ingredients, it is necessary to restrict the operation of the aerosol generating device 200 for a certain amount of time to prevent misuse or abuse by the user. When the cartridge liquid information includes information on an interval between two consecutive inhalations, the terminal may restrict operation of the aerosol generating device 200 before the next inhalation start time according to the inhalation completion notification, and update the operating condition information of the electronic device to set the next inhalation start time.

The terminal may determine a next inhalation start time of the cartridge liquid, and may restrict the operation of the aerosol generating device 200 before the determined next inhalation start time. For example, the terminal may control the aerosol generating device 200 not to operate during a certain period of time to prevent excessive inhalation of a specific liquid component (e.g., the mind and body stability and/or the pharmacological ingredients) after the completion of one inhalation cycle (i.e., one dose). When the user tries to use the aerosol generating device 200 before the next inhalation start time, the terminal may transmit a notification signal indicating that the operation is not possible to the aerosol generating device 200. The aerosol generating device 200 which has received the notification signal may provide a notification indicating that the operation is not possible through an interface. In addition, the terminal itself may provide the notification indicating that the operation of the aerosol generating device 200 is not possible through its own interface.

Furthermore, when a next inhalation cycle (i.e., inhalation start time) is imminent, the terminal may transmit a notification signal for notifying the inhalation time to the aerosol generating device 200. The aerosol generating device 200 which has received the notification signal may provide a notification for notifying that the inhalation is available, through the interface. In addition, the terminal itself may provide the notification for the inhalation time through the interface. The notification for the inhalation time may be provided, not only at the inhalation start time, but also a predetermined time before the inhalation start time. For example, the terminal may provide a guide message of “There is 1 hour left until the next inhalation start time.”

According to an embodiment, the terminal may communicate with the electronic device through the short-range wireless communication unit 182 (see FIG. 1), and communicate with the server through the wireless communication unit 184 (see FIG. 1).

FIG. 6 is a flowchart illustrating a method of controlling an aerosol generating device according to an embodiment.

For ease of description, it will be described that operations 611 to 615 below are performed by the aerosol generating device 200 shown in FIG. 2. However, operations 611 to 615 may be performed by another suitable electronic device in a suitable system.

Furthermore, the operations of FIG. 6 may be performed in the shown order and manner. However, the order of some operations may change, or some operations may be omitted, without departing from the spirit and scope of the shown example. The operations shown in FIG. 6 may be performed in parallel or simultaneously. Hereinafter, the aerosol generating device 200 may be referred to as an electronic cigarette or an electronic device. The description provided with reference to FIG. 5 may apply to FIG. 6, and any repeated description related thereto may be omitted.

In operation 611, the aerosol generating device 200 may determine the type of a liquid included in a cartridge liquid. For example, when the insertion detection sensor 124 of the aerosol generating device 200 detects the insertion of a cartridge, the controller 214 of the aerosol generating device 200 may identify the type of the cartridge liquid by identifying a composition of the cartridge liquid (e.g., through a temperature profile) or by using a predetermined impedance value of the cartridge liquid.

In operation 612, the aerosol generating device 200 may transmit a liquid type signal to a terminal connected to the aerosol generating device 200. The liquid type signal may indicate the liquid type of the cartridge determined in operation 611. The terminal may be the terminal described above with reference to FIG. 5.

In operation 613, the aerosol generating device 200 may receive a control signal including information about the liquid, which is obtained by the terminal based on the liquid type, from the terminal. The information about the liquid may indicate at least one of the puffing time, the puffing cycle, and the puffing number, which are required for one dose of the liquid.

In operation 614, the aerosol generating device 200 may control an operation of the aerosol generating device 200 based on the control signal. The aerosol generating device 200 may provide a puff-inducing interface to the user based on the control signal. The operation of the aerosol generating device 200 may be started in response to a signal indicating that the user authentication is completed.

For example, when the control signal includes information on the puffing time, the puffing cycle, and the puffing number required for one dose, the information may be displayed on the display 132 of the aerosol generating device 200. In addition, the haptic portion 134 may operate at each puffing time and puffing cycle in order to induce the user to properly inhale.

In operation 615, the aerosol generating device 200 may transmit an inhalation completion notification to the terminal. For example, when the inhalation of the user is completed according to the control signal including the information about the liquid, the aerosol generating device 200 may stop generating an aerosol and transmit a signal indicating that the inhalation of the user is completed to the terminal.

The embodiments described herein may be implemented using a hardware component, a software component and/or a combination thereof. A processing device may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor or any other device capable of responding to and executing instructions in a defined manner. The processing device may run an OS and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described devices may be configured to act as one or more software modules in order to perform the operations of the above-described embodiments, or vice versa.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Therefore, other implementations, other embodiments, and equivalents to the claims are also within the scope of the following claims.

1. A method of controlling a terminal, the method comprising:
receiving, from an electronic device, information about a cartridge liquid of the electronic device;
transmitting a request for information about the cartridge liquid to a server;
receiving the information about the cartridge liquid from the server;
transmitting a control signal comprising the information about the cartridge liquid to the electronic device;
receiving an inhalation completion notification from the electronic device; and
updating operating condition information about the electronic device in response to the inhalation completion notification.

2. The method of claim 1, further comprising:
displaying the information about the cartridge liquid on the terminal.

3. The method of claim 1, wherein the updating of the operating condition information comprises:
determining a next inhalation start time of the cartridge liquid; and restricting an
operation of the electronic device until the determined next inhalation start time.

4. The method of claim 1, further comprising:
performing user authentication when the information about the cartridge liquid comprises information about inhalation restriction.

5. The method of claim 4, wherein the performing of the user authentication comprises:
controlling the electronic device to cease the operation until the user authentication is completed.

6. The method of claim 1, wherein the information about the cartridge liquid indicates at least one of a puffing time, a puffing cycle, and a number of puffs, which are required for one dose of the cartridge liquid.

7. A method of controlling an electronic device, the method comprising:
determining a type of a liquid included in a cartridge;
transmitting a signal identifying the type of the liquid to a terminal;
in response to the signal, receiving a control signal comprising information about the liquid from the terminal;
controlling an operation of the electronic device based on the control signal; and
transmitting an inhalation completion notification to the terminal when inhalation of one dose of the liquid is completed.

8. The method of claim 7, wherein the controlling of the operation of the electronic device comprises:
providing a puff-inducing interface based on the control signal.

9. A non-transitory computer-readable storage medium storing instructions that, when executed by a processor, cause the processor to perform the method of claim 1.

10. A terminal comprising:
a short-range wireless communication unit configured to receive information about a cartridge liquid of an electronic device from the electronic device, and receive an inhalation completion notification from the electronic device;
a wireless communication unit configured to transmit a request for information about the cartridge liquid to a server and receive the information about the cartridge liquid from the server; and
a controller configured to update operating condition information about the electronic device in response to the inhalation completion notification,
wherein the short-range wireless communication unit is configured to transmit a control signal comprising the information about the cartridge liquid to the electronic device.

11. (canceled)
